(19) United States
(12) Patent Application Publication
(10) Pub. No.: US 2018/0325586 A1
(43) Pub. Date: Nov. 15, 2018

(54) SINGLE CATHETER FOR CARDIAC ABLATION AND MAPPING

(71) Applicant: Boston Scientific Scimed Inc., Maple Grove, MN (US)

(72) Inventors: Brian Stewart, North Reading, MA (US); Nathan H. Bennett, Cambridge, MA (US); Vasiliy E. Buharin, Cambridge, MA (US); Charles A. Gibson, Malden, MA (US); Kurt E. Guggenberger, North Andover, MA (US); William Quinn, Swampscott, MA (US)

(21) Appl. No.: 15/976,400

(22) Filed: May 10, 2018

Related U.S. Application Data
(60) Provisional application No. 62/504,249, filed on May 10, 2017.

(51) Int. Cl. A61B 18/14; A61M 25/00

(52) U.S. Cl. CPC A61B 18/1492; A61M 25/0082; A61B 2018/00577; A61B 2562/043; A61M 2210/125; A61B 2018/1467

(57) ABSTRACT

An ablation catheter comprises a shaft having a proximal end portion, and a distal end portion having a distal end and defining a longitudinal axis of the ablation catheter. An ablation electrode is located at the distal end of the shaft. A mapping region including a plurality of mini-electrode sets disposed about the shaft is located proximal to the ablation electrode. Each of the mini-electrode sets includes a plurality of mini-electrodes. The ablation catheter further includes a deflection region proximate to or within the mapping region.

### CROSS-REFERENCE TO RELATED APPLICATION

This application claims priority to Provisional Application No. 62/504,249, filed May 10, 2017, which is herein incorporated by reference in its entirety.

### TECHNICAL FIELD

The present invention relates to medical devices and methods for treating cardiac arrhythmias. More specifically, the invention relates to devices and methods for performing cardiac ablation for terminating left atrial fibrillation and the like.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In Example 1, an ablation catheter comprising a shaft, an ablation electrode and a mapping region. The shaft has a proximal end portion, and a distal end portion having a distal end and defining a longitudinal axis of the ablation catheter. The ablation electrode is located at the distal end of the shaft. The mapping region includes a plurality of mini-electrode sets disposed about the shaft proximal to the ablation electrode, wherein each of the mini-electrode sets includes a plurality of mini-electrodes.

In Example 2, the ablation catheter of Example 1, wherein each of the mini-electrode sets includes a plurality of mini-electrodes arranged along a line generally parallel to the longitudinal axis, and wherein the mini-electrode sets are disposed circumferentially about the shaft.

In Example 3, the ablation catheter of any of Examples 1-2, wherein the plurality of mini-electrode sets consists of three mini-electrode sets equally circumferentially spaced about the shaft.

In Example 4, the ablation catheter of any of Examples 1-2, wherein the plurality of mini-electrode sets consists four mini-electrode sets equally circumferentially spaced about the shaft.

In Example 5, the ablation catheter of any of Examples 1-4, wherein each of the plurality of mini-electrode sets comprises 2-8 mini-electrodes each located at a respective longitudinal position along the shaft, and wherein two of the mini-electrode sets have the same number of mini-electrodes.

In Example 6, the ablation catheter of any of Examples 1-5, wherein the mini-electrodes of each mini-electrode set are longitudinally spaced at a center-to-center spacing of from 0.5 millimeters to 2 millimeters.

In Example 7, the ablation catheter of any of Examples 1-6, wherein the mini-electrodes are rectangular in shape.

In Example 8, the ablation catheter of Example 1, wherein the mini-electrode sets are configured in the form of bands longitudinally spaced along the mapping region, wherein the mini-electrodes of each mini-electrode set are circumferentially spaced about the band.

In Example 9, the ablation catheter of any of Examples 1-8, wherein each mini-electrode of each mini-electrode set is disposed on a flexible circuit.

In Example 10, the ablation catheter of any of Examples 1-9, wherein the mini-electrodes have an active surface area of between 0.2 mm² to 1 mm².

In Example 11, the ablation catheter of any of Examples 1-9, wherein the mini-electrodes each have an active surface area sized for maximum signal attainment.

In Example 12, the ablation catheter of any of Examples 1-11, wherein the mini-electrodes have a rounded or generally spherically-shaped active region.

In Example 13, the ablation catheter of any of Examples 1-12 wherein the insulative material is overmolded about the mini-electrode sets so as to provide for active regions of each of the mini-electrodes.

In Example 14, the ablation catheter of Example 1-13, wherein the shaft includes a first deflection region at which the shaft is configured to bend in a pre-determined direction, wherein the first deflection region is located proximal to or within the mapping region and is configured to bend in a first direction.

In Example 15, the ablation catheter of Example 14, wherein the shaft further includes a second deflection region located distally of the first deflection region, the second deflection region configured to bend in a second direction different than the first direction.

In Example 16, an ablation catheter comprising a shaft, an ablation electrode and a mapping region. The shaft has a proximal end portion, and a distal end portion having a distal end and defining a longitudinal axis of the ablation catheter, and the shaft is configured to include a first deflection region at which the shaft is configured to bend in a first pre-determined direction. The ablation electrode is located at the distal end of the body. The mapping region includes a plurality of mini-electrode sets disposed about the shaft proximal to the ablation electrode, each of the mini-electrode sets including a plurality of mini-electrodes. The first deflection region is located proximal to or within the mapping region.

In Example 17, the ablation catheter of Example 16, wherein each of the mini-electrode sets includes a plurality of mini-electrodes arranged along a line generally parallel to the longitudinal axis, and wherein the mini-electrode sets are disposed circumferentially about the shaft.

In Example 18, the ablation catheter of Example 17, wherein the plurality of mini-electrode sets consists of three mini-electrode sets equally circumferentially spaced about the shaft.

In Example 19, the ablation catheter of Example 17, wherein the plurality of mini-electrode sets consists four mini-electrode sets equally circumferentially spaced about the shaft.

In Example 20, the ablation catheter of Example 17, wherein each of the plurality of mini-electrode sets comprises 2-8 mini-electrodes each located at a respective longitudinal position along the shaft, and wherein two of the mini-electrode sets have the same number of mini-electrodes.

In Example 21, the ablation catheter Example 17, wherein the mini-electrodes of each mini-electrode set are longitudinally spaced at a center-to-center spacing of from 0.5 millimeters to 2 millimeters.

In Example 22, the ablation catheter of Example 17, wherein the mini-electrode sets are configured in the form of bands longitudinally spaced along the mapping region, wherein the mini-electrodes of each mini-electrode set are circumferentially spaced about the band.

In Example 23, the ablation catheter of Example 17, wherein each mini-electrode of each mini-electrode set is disposed on a flexible circuit.

In Example 24, the ablation catheter of Example 17, wherein the mini-electrodes have an active surface area of between 0.2 mm² to 1 mm².

In Example 25, the ablation catheter of Example 17, wherein the shaft further includes a second deflection region located distally of the first deflection region, the second deflection region configured to bend in a second direction different than the first direction.

In Example 26, a medical method comprising advancing an ablation electrode disposed at a distal end portion of a shaft of an ablation catheter to a left atrial location proximate an ostium of a pulmonary vein of a patient's heart, wherein the distal end portion includes a plurality of mini-electrode sets disposed circumferentially about the shaft proximal to the ablation electrode, each of the mini-electrode sets including a plurality of mini-electrodes arranged therein. The method further comprises then applying ablation energy using the ablation electrode to tissue proximate the ostium with so as to form a conduction block within the tissue, and after terminating the application of the ablation energy, advancing the distal end portion further into the pulmonary vein. The method further comprises then causing at least some of the mini-electrodes to be urged into contact with the ablated tissue, acquiring signals from the mini-electrodes in contact with the ablated tissue, and based on the signals, analyzing the extent of the conduction block.

In Example 27, the method of Example 26, further comprising acquiring a three-dimensional electroanatomical map of the left atrium, acquiring positional information for the distal end portion of the ablation catheter, and displaying the position of the distal end portion of the ablation catheter on the electroanatomical map during one or both of advancing the ablation electrode and applying the ablation energy.

In Example 28, the method of Example 27, further comprising updating the electroanatomical map based at least in part on the signals acquired from the mini-electrodes after terminating the ablation energy.

In Example 29, the method of Example 28, wherein causing at least some of the mini-electrodes to be urged into contact with the ablated tissue includes forming a bend in the distal end portion of the shaft.

In Example 30, the method of Example 29, wherein causing at least some of the mini-electrodes to be urged into contact with the ablated tissue further includes moving the ablation catheter so as to sweep at least some of the mini-electrodes about substantially the entire circumference of the tissue proximate the ostium.

In Example 31, the method of Example 28, further comprising re-applying ablation energy to tissue proximate the ostium if the analysis of the signals indicates a gap in the conduction block.

In Example 32, a medical system comprising a mapping system, an ablation energy source, and an ablation catheter. The mapping system is configured to generate a three-dimensional anatomical map of a cardiac chamber of interest. The ablation energy source is configured to provide ablation energy for a cardiac ablation procedure. The ablation catheter is operatively coupled to the mapping system and the ablation energy source, and includes a shaft, an ablation electrode, and a mapping region. The shaft has a proximal end portion, and a distal end portion having a distal end and defining a longitudinal axis of the ablation catheter, and is configured to include a first deflection region at which the shaft is configured to bend in a first pre-determined direction. The ablation electrode is located at the distal end of the body and is operatively coupled to the ablation energy source. The mapping region includes a plurality of mini-electrode sets disposed about the shaft proximal to the ablation electrode, wherein each of the mini-electrode sets includes a plurality of mini-electrodes operatively coupled to the mapping system. The first deflection region is located proximal to or within the mapping region.

In Example 33, the medical system of Example 32, wherein each of the mini-electrode sets includes a plurality of mini-electrodes arranged along a line generally parallel to the longitudinal axis, and wherein the mini-electrode sets are disposed circumferentially about the shaft.

In Example 34, the medical system of Example 32, wherein the mini-electrode sets are configured in the form of bands longitudinally spaced along the mapping region, wherein the mini-electrodes of each mini-electrode set are circumferentially spaced about the band.

In Example 35, the medical system of Example 32, wherein the mini-electrodes have an active surface area of between 0.2 mm² to 1 mm².

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic elevation view of an ablation and mapping system in an embodiment of the present invention.

FIGS. 2-4 are partial isometric illustrations of the distal end portion of alternative embodiments of an ablation catheter usable in the ablation and mapping system of FIG. 1.

FIG. 5 is a schematic illustration of the use of an ablation catheter according to embodiments of the invention to perform a pulmonary vein isolation procedure.

FIG. 6 is a schematic illustration of the use of an ablation catheter according to embodiments of the invention to map tissue proximate the pulmonary vein ostium following a pulmonary vein isolation procedure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

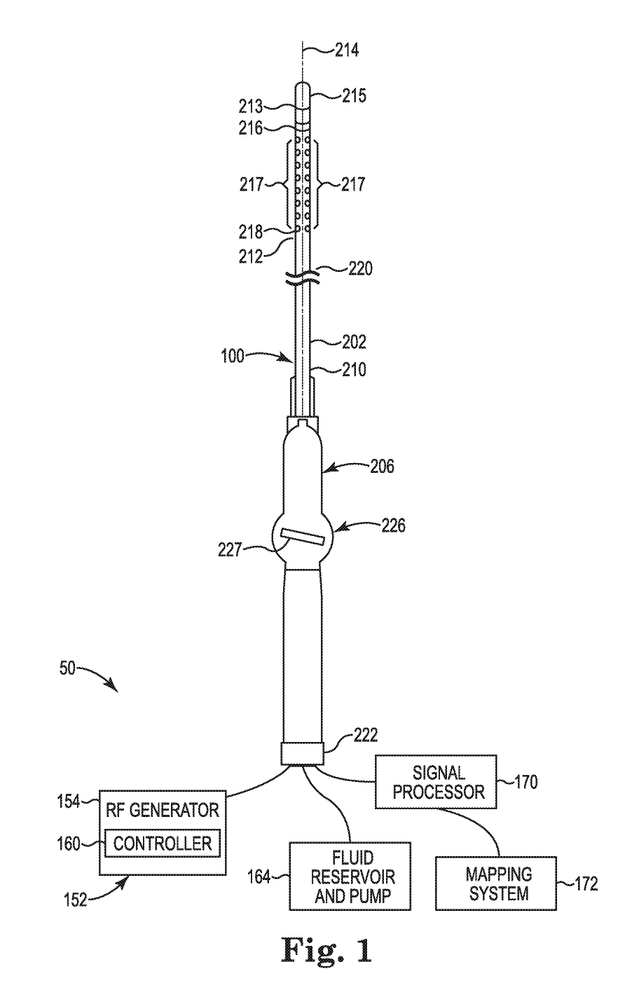

FIG. 1 is a schematic elevation view of an ablation and mapping system 50 including the catheter device 100, in an embodiment of the present invention. As will be explained in greater detail elsewhere herein, the system 50 can be particularly useful in performing ablation procedures within the cardiac chambers to treat cardiac arrhythmias. In one exemplary embodiment, the system 50 is used to perform pulmonary vein isolation procedures, whereby arrhythmogenic tissue about or proximate the ostia of the pulmonary veins are ablated to form complete conduction blocks. Additionally, the system 50 may be used to perform and validate ablation procedures along the left atrial walls or other cardiac chambers, e.g., the right atrium or the ventricle(s).

As shown in FIG. 1, in addition to the catheter device 100, the system 50 includes additional hardware and equipment including, in the particular embodiment shown, an ablation control system 152 including a radiofrequency generator 154 coupled to a controller 160, a fluid delivery system 164 including, among other things, a fluid reservoir and pump, a signal processor 170 and a mapping system 172. In various embodiments, for example, a system incorporating a non-irrigated catheter device 100, the fluid delivery system 164 can be omitted. In various embodiments, the ablation control system 152 is configured to provide a controlled amount of RF energy to the catheter device 100 as needed for the particular ablation procedure being performed. The processor 160 controls the timing and the level of the RF energy delivered through the catheter device 100.

In the various embodiments, the signal processor 170 is configured, at least in part, to receive and process cardiac signals obtained by the catheter device 100 for interpretation and use by the clinician during the ablation procedure and, in various embodiments, to be outputted to the mapping system 172. The signal processor 170 can be configured to detect, process, and record electrical signals within the heart 104. Based on the detected electrical signals, the signal processor 170 outputs electrocardiograms (ECGs) the mapping system 172 which includes a display (not shown) that can provide to the user a variety of display windows and images, including without limitation, a three-dimensional electroanatomical map of the cardiac region of interest.

In various embodiments, the mapping system 172 can be any cardiac mapping system, whether now known or later developed. In one embodiment, the mapping system 172 can be any generation of the Rhythma™ mapping system marketed by Boston Scientific Corporation. The Although the ablation control system 152, the fluid delivery system 164, and the signal processor 170 are shown as discrete components, they can alternatively be incorporated into a single integrated device.

It is emphasized that the particular configuration and presence of the ablation control system 152, the fluid delivery system 164 and the signal processor 170 are not critical to the various embodiments. Thus, when present, any such systems and hardware, whether currently known or later developed, can be utilized within the system 50.

As further shown in FIG. 1, the catheter 100 includes a shaft 202, a handle 206, and a control mechanism 208. Additionally, the shaft 202 includes a proximal end portion 210, and an opposite distal end portion 212 having a distal end 213, and defines a longitudinal axis 214 of the catheter 100. As further shown, a tip electrode 215 is disposed at the distal end 213 of the shaft 202, and a ring electrode 216 is disposed about the shaft 202 proximal to the tip electrode 215. Additionally, a plurality of mini-electrode sets 217 each comprising a plurality of mini-electrodes 218 are disposed circumferentially about the shaft 202 proximal to the ring electrode 216. As will be explained in greater detail elsewhere herein, the mini-electrode sets 217 are configured for high-density acquisition of electrical signals within the cardiac chamber of interest, and thus the presence of the mini-electrode sets 217 defines a mapping region of the shaft 202 of the catheter 100.

Furthermore, in the illustrated embodiment, the shaft 202 has a deflection region 220 located in proximity to the mapping region of the shaft 202. In the illustrated embodiment, the deflection region 220 is located proximal to the proximal-most of the mini-electrodes 218. although in other embodiments the deflection region 220 can be located more distally than as shown, including longitudinally within the mapping region of the shaft 202. The configuration and functionality of the mini-electrode sets 217 and the deflection region 220 will be discussed in greater detail elsewhere herein.

In various embodiments, the elongate shaft 202 is generally tubular and houses additional components including, without limitation, electrical conductors and, as will be discussed in greater detail below, components for manipulating the catheter device 100, including the tip section 204, during the ablation procedures.

In various embodiments, the shaft 202 can be formed of an inert, resilient polymeric material that retains its shape and does not soften significantly at body temperature; for example, polyether block amides, polyurethane, polyester, and the like. The shaft 202 can be flexible so that it is capable of winding through a tortuous path that leads to a target site. In some embodiments, the shaft 202 can be reinforced with a coating, braid, coil, or similar structure, to control the flexibility and torqueability of the shaft 202.

As further shown, the handle 206 is coupled to the proximal end portion 210 of the shaft 202, and includes a connection port 222, and a portion of the control mechanism 208 (in the illustrated embodiment, a control element 226). The connection port 222 is operable to allow external devices and hardware, e.g., the ablation control system 152, the fluid delivery system 164 and/or the signal processor 170, to be operably coupled to the catheter device 100. In addition, the handle 206 further includes a plurality of conduits, conductors, and wires (not shown) to facilitate control of the catheter device 100. In the illustrated embodiment, the control element 226 includes a control knob 227 operably to be manipulated by the clinician to deflect the distal end 212 of the shaft 202. As such, the control knob 227 is mechanically and operably coupled to additional components (e.g., one or more control wires) extending along the shaft 202. It is emphasized, however, that the particular mechanism for controlling deflection and steerability of the catheter device 100 is not critical to the various embodiments of the present invention. In addition, in various embodiments, the catheter device 100 is a fixed-shape catheter (i.e., is not steerable) and thus the control knob 227 and associated components can be omitted in such embodiments.

The tip electrode 215 is formed from an electrically conductive material and is operable as an ablation electrode for delivering ablation energy (e.g., RF energy) to the tissue of interest. In some embodiments may use a platinum-iridium alloy. Some embodiments may use an alloy with approximately 90% platinum and 10% iridium. However, in other embodiments, other materials, e.g., titanium and alloys thereof, are used for the tip electrode 215.

The ring electrode 216 is operable to facilitate the acquisition of cardiac electrogram signals, among other things as will be appreciated by those skilled in the art. In the illustrated embodiment, a single ring electrode 216 is present, while in other embodiments additional ring electrodes 216 can be incorporated, or alternatively, the catheter 100 may include no ring electrodes.

In various embodiments, the catheter 100 can include a plurality of mapping electrodes (not shown) located within the tip electrode 215 to facilitate high-fidelity sensing of localized electrical signals at the location of the tip electrode 215. In some embodiments, the catheter 100 can be constructed according to and include the features and functionality of any of the embodiments disclosed in commonly-assigned U.S. Pat. No. 8,414,579 and in commonly-assigned U.S. Patent Application Publication 2015/0133914, the disclosures of which are each incorporated by reference herein in their entireties.

In some embodiments, the catheter 100 can include additional components to enhance its functionality. For example, the catheter 100 can include navigation sensors (e.g., electromagnetic coil or magneto-resistive sensors) to facilitate accurate localization of the distal end portion 212 within the subject anatomy. Additionally, the catheter 100 can include contact sensors or force sensors to provide for an indication of contact between the tip electrode 215 and the subject tissue, or the magnitude and direction of force applied to the tissue by the tip electrode 215, as the case may be. In one embodiment, the catheter 100 may include a force sensing arrangement such as described in any of the co-pending and commonly-assigned U.S. Provisional Patent Application 62/270,016 filed on Dec. 20, 2015, the disclosure of which are is incorporated by reference herein in its entirety.

The mini-electrode sets 217 are circumferentially spaced about the longitudinal axis 214 of the shaft 202. As can be seen in FIG. 1, the mini-electrodes 218 of each mini-electrode set 217 are arranged along a line generally parallel to the longitudinal axis 214. The mini-electrode sets 217 are configured for highly sensitive localized mapping of the cardiac tissue.

The inclusion of the mini-electrode sets 217 can be particularly advantageous in cardiac ablation procedures, such as procedures to isolate the pulmonary veins for treatment of atrial fibrillation. In particular, as will be explained in further detail elsewhere herein, the mini-electrodes 217 can facilitate high-density mapping of target tissue both before and after the application of ablation energy using the same catheter through which the ablation energy itself is applied.

The deflection region 220 is configured to bend in a pre-determined direction, e.g., by manipulation of the control element 226, so as to change the trajectory of the distal end portion 212 of the shaft 202, and in particular, the mapping region thereof containing the mini-electrode sets 217. This ability to change the shape of the shaft 202 within or proximate the mapping region is useful in allowing the user to urge the mini-electrodes 218 into direct contact with target tissue, particularly when the target tissue is within a body lumen such as a pulmonary vein.

The deflection region 220, and the means for effectuating the deflection of the distal end portion 212, can be implemented by any techniques known, or later developed, for deflectable catheters or other medical probes. As will be appreciated, steerable catheters generally are well known in the art, as are a variety of mechanisms to effectuate the deflection of the various portions of the catheters. In one exemplary embodiment, the deflection region 220 and the associated curve therein can be achieved through independent steering components such as those described in commonly-owned U.S. Patent 8,007,462, the disclosure of which is incorporated herein by reference in its entirety for all purposes. In particular, the aforementioned U.S. Pat. No. 8,007,462 discloses various embodiments for providing multiple, independently-actuatable bends in a catheter body, which may be readily employed in the ablation catheter 100. Thus in some embodiments, the ablation catheter 100 includes one or more additional deflection region(s) located distally of the deflection region 220, any of which may be implemented using the teachings of the aforementioned U.S. Pat. No. 8,007,462, so that the shaft 202 can exhibit compound curvature defined by multiple bends.

In other embodiments, other techniques may be employed to provide the desired deflection regions and corresponding bends. For example, in embodiments, the shaft 202 may include one or more fixed or preformed curves at the deflection region 220 (or additional deflection regions where present). In such embodiments, deflection region 220 may be partially straightened (e.g., using a stylet, guidewire, or similar device) during advancement of the distal end portion 212 to the left atrium, and then allowed to resume its preformed bend upon removal of the straightening means when at the target location. In short, the particular technique and structure used for effectuating the bend in the deflection region 220 and/or the distal end 213 in the various embodiments is not of critical importance.

It is noted that the deflection region 220 may be present in addition to or in lieu of additional steering features that may be present to, for example, deflect the distal end 213 to facilitate navigation of the distal end 213 through the patient's anatomy.

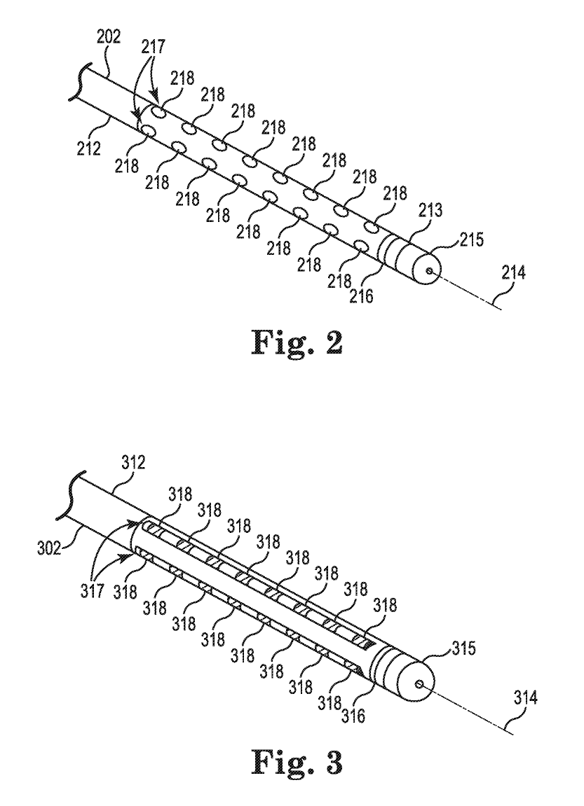

FIG. 2 is a partial isometric illustration of the distal end portion 212 the shaft 202 of the ablation catheter 100 according to an embodiment. As shown in FIG. 2, each of the mini-electrode sets 217 is disposed such that its mini-electrodes 218 are arranged in a line generally parallel to the longitudinal axis 214. As further shown, electrically insulative material of the shaft 202 is disposed between the mini-electrodes 218 so as to electrically isolate the mini-electrodes 218 from one another. In various embodiments, the various mini-electrode sets 217 can be arranged such that they are equally spaced from one another about the circumference of the shaft 202.

In the illustrated embodiment, the mini-electrodes 217 have an active region (i.e., the region exposed to patient tissue in use) that is spherically-shaped. In other embodiments, the mini-electrodes 218 can have other geometries.

FIG. 3 is a partial isometric illustration of an alternative ablation catheter 300 according to another embodiment. The ablation catheter 300 is readily adaptable for use in the system 50 of FIG. 1 in place of the ablation catheter 100. As shown, the ablation catheter 300 has a shaft 302 having a distal end portion 312 with a distal end 313, and defines a longitudinal axis 314 of the catheter 300. As further shown, a tip electrode 315 is disposed at the distal end 313 of the shaft 302, and a ring electrode 316 is disposed about the shaft 302 proximal to the tip electrode 315. Additionally, a plurality of mini-electrode sets 317 each comprising a plurality of mini-electrodes 318 are disposed circumferentially about the shaft 302 proximal to the ring electrode 316. As with the catheter 100, the location of the mini-electrode sets 317 defines a mapping region of the shaft 302 of the catheter 300.

The ablation catheter 300 differs from the ablation catheter 100 in that the mini-electrodes 318 are generally rectangular in shape, with a long dimension generally oriented parallel with the longitudinal axis 314. As shown, the mini-electrodes 318 have a rounded profile, although in other embodiments the outer surfaces of the mini-electrodes may have other profiles (e.g., substantially flat). The ablation catheters 100, 300 have, in other respects, substantially the same design and functionality.

The specific configuration, size, position, and number of mini-electrodes of the mini-electrode sets 217, 317 can vary from catheter to catheter depending on the specific clinical intent. In embodiments, the mini-electrodes 218, 318 can have an active surface area of 0.2 mm² to 1 mm². In general, the axial length of the mini-electrode sets 217, 317 and the active surface area of the mini-electrodes 217, 317 can be sized to provide for maximum signal attainment within the cardiac chamber, in particular, the left atrium.

In various embodiments, the mini-electrodes 218, 318 are equally spaced along the length of the respective mini-electrode sets 217, 317 at a center-to-center spacing of 0.5 millimeters to 3 millimeters between neighboring mini-electrodes 218, 318. In other embodiments, however, the mini-electrodes 218, 318 are unequally spaced along the length of the respective mini-electrode sets 217, 317, and/or the mini-electrode spacing can differ among the respective mini-electrode sets 217, 317.

As previously stated, the number of mini-electrode sets 217, 317 can be selected based on the particular desired functionality. In various embodiments, the ablation catheters 100, 300 can have between two and four mini-electrode sets 217, 318 disposed about the circumference of the respective catheter shaft 202, 302. In other embodiments, more than four mini-electrode sets 217, 317 can be employed.

Similarly, the number of mini-electrodes 218, 318 in each set can be varied from catheter to catheter. In the illustrated embodiments, the mini-electrode sets 217, 318 each have, respectively eight equally-spaced mini-electrodes 218, 318. In other embodiments, the mini-electrode sets 217, 317 may have as few as two or more than eight mini-electrodes 218, 318.

In some embodiments, such as shown in FIG. 3, portions of the respective mini-electrodes 218, 318 may be masked or otherwise covered such that only the exposed portions of the mini-electrodes 218, 318 are exposed to the external environment. This configuration may advantageously provide for directional mapping capability.

FIG. 4 is a partial isometric illustration of an alternative ablation catheter 350 according to another embodiment. The ablation catheter 350 is readily adaptable for use in the system 50 of FIG. 1 in place of the ablation catheter 100. As shown, the ablation catheter 350 has a shaft 351 having a distal end portion 352 with a distal end 353, and defines a longitudinal axis 354 of the catheter 350. As further shown, a tip electrode 355 is disposed at the distal end 353 of the shaft 351, and a ring electrode 356 is disposed about the shaft 351 proximal to the tip electrode 355. Additionally, a plurality of mini-electrode sets 357 each comprising a plurality of mini-electrodes 358 are disposed circumferentially about the shaft 351 proximal to the ring electrode 356. As with the catheters 100 and 300, the location of the mini-electrode sets 357 defines a mapping region of the shaft 351 of the catheter 350.

The ablation catheter 350 differs from the ablation catheters 100 and 200 in that the mini-electrode sets 357 are configured in the form of circumferential bands that are longitudinally-spaced along a length of the distal end portion 352 of the shaft 351. As shown in FIG. 4, the mini-electrodes 358 of each mini-electrode set 357 are circumferentially spaced about the band. In some embodiments, the mini-electrodes 358 are uniformly spaced about the band, whereas in other embodiments, the mini-electrodes may be unequally spaced depending on the particular operational requirements of the catheter 350.

The particular construction techniques used to form and incorporate the mini-electrode sets 217, 317 and 357 is not critical to the present disclosure. In one embodiment, the mini-electrodes 218, 318 and 358 can be formed on flex circuits that may comprise one or more than one of the mini-electrode sets 217, 317 or 357 on the respective catheter. In one embodiment, each mini-electrode 218, 318 or 358 is discretely disposed within a support material of the respective shaft 202, 302. In embodiments, the insulative material of the shaft 202, 302, 351 in which the mini-electrode sets 217, 317, 357 may be formed by overmolding techniques as are known in the art. However, any number of medical device and electrical component construction techniques may be utilized within the scope of the present disclosure.

The inclusion of the mini-electrode sets 217, 317 and 357 may enhance the speed and efficiency of cardiac ablation procedures, and in particular, pulmonary vein isolation procedures to treat left atrial fibrillation. Conventionally, these procedures are often performed using guidance provided by a pre-acquired three-dimensional electroanatomical map of the left atrium, e.g., such as those provided by the Rhythmia™ Mapping System of Boston Scientific. After applying the ablation energy to the target tissue, e.g., tissue proximate the pulmonary vein ostia, the region must be re-mapped so that the physician can ascertain whether the ablation has created an effective conduction block. The effectiveness of conventional ablation catheters for use in this re-mapping process is limited due to the large size and overall configuration of the sensing electrodes on such catheters. Alternatively, the use of a multi-electrode mapping catheter, e.g., the ORION™ mapping catheter marketed by Boston Scientific Corporation, requires retraction of the ablation catheter from the treatment site, and in some cases, from the left atrium entirely. However, with the ablation catheters 100, 300 of the present disclosure, the physician can more rapidly perform high-density localized mapping of the ablated tissue using the same catheter used to perform the ablation itself.

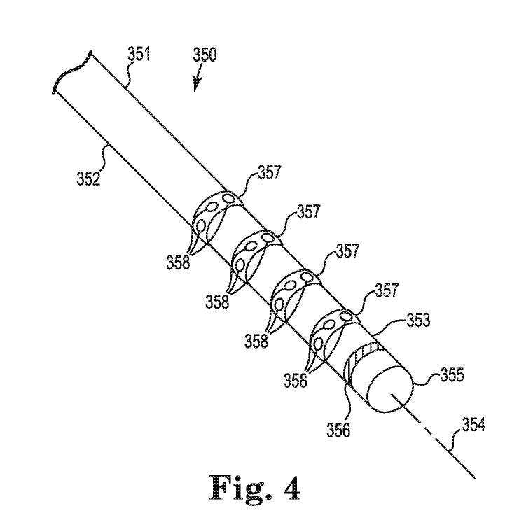

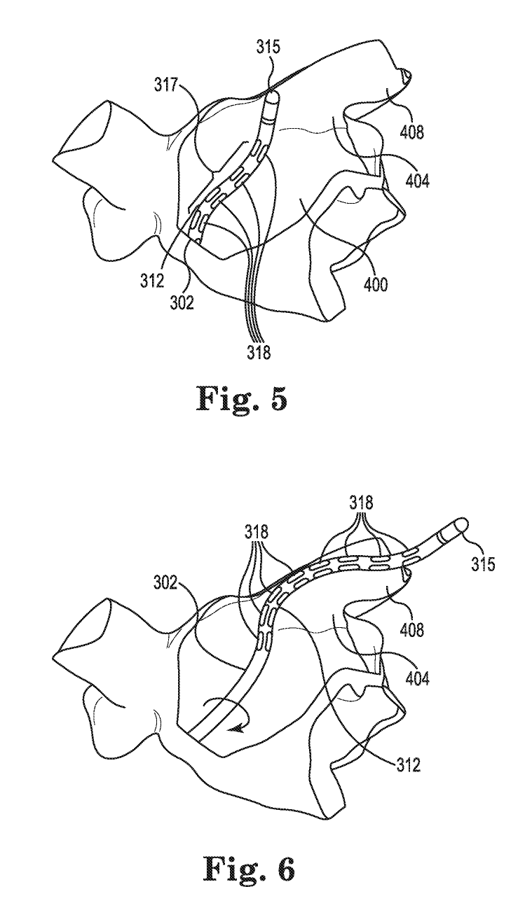

The foregoing is illustrated schematically in FIGS. 5-6, showing the use of an ablation catheter according to embodiments of the invention (in this case, the ablation catheter 300) to perform a pulmonary vein isolation procedure within a left atrium 400 and subsequent re-mapping of the ablated region. In an embodiment, prior to performing the ablation procedure, a three-dimensional electroanatomical map (not shown) of the left atrium is acquired. Additionally, in various embodiments, positional information for at least the distal end portion 312 of the catheter 300 can be acquired during the ablation procedure, so that the position of the distal end portion 312 is displayed on the electroanatomical map during the procedure.

As shown in FIG. 5, the tip electrode 315 is advanced to a location within the left atrium 400 proximate an ostium 404 of a pulmonary vein 408, and in contact with the target tissue to be ablated. Specific techniques for performing the pulmonary vein isolation are well known and need not be described in great detail herein. Generally speaking, the physician applies ablation energy to the target tissue via the tip electrode 315, and sequentially repositions the tip electrode about the target region so as to form a therapeutically effective conduction block in the tissue.

As shown in FIG. 6, after terminating the application of the ablation energy, the distal end portion 312 can be advanced further into the pulmonary vein 408 so that at least some portion of the shaft 302 having the mini-electrode sets 317 is located proximate the ablated region. As further shown, the distal end portion 312 can then be deflected (e.g., via a deflection region similar to the deflection region 220 of the ablation catheter 100). Deflection of the distal end portion 312 in such a manner can facilitate the physician urging the mini-electrodes 318 into contact with the ablated tissue (as well as adjacent tissue). The physician can then move the ablation catheter 300 in such a manner so as to sweep the mini-electrodes 318 about substantially the entire circumference of the ablated region and thereby acquire signals indicative of the tissue's conductive properties post-ablation.

These acquired post-ablation signals from the mini-electrodes 318 can then be utilized to update the electroanatomical map. In the event the updated electroanatomical map indicates an incomplete or ineffective conduction block has been formed (e.g., there is a gap in the lesion), the physician can then re-position the tip electrode 315 (using the updated electroanatomical map for guidance) and re-apply ablation energy as appropriate.

If appropriate, upon completion of the procedure at one pulmonary vein ostium, the procedures described above can be repeated at one or more of the additional pulmonary vein ostia.

In other embodiments, the ablation catheters of the present disclosure can be used to perform and validate ablation procedures in addition to pulmonary vein isolation procedures. For example, in one embodiment, the catheter 300 (as an example) can be used to form generally linear ablation lines along selected tissue of the left atrial wall. Subsequently, the physician can cause deflection of the distal end portion 312 in such a manner so as to facilitate urging the mini-electrodes 318 into contact with the ablated tissue and adjacent tissue. The physician can then sweep the mini-electrodes 318 along the left atrial wall to generate high-density electroanatomical information, which can be used to assess whether the ablation has formed a complete conduction block. Advantageously, the catheter 300 allows the physician to perform both the ablation and the conduction block validation using the same catheter. Similar procedures can also be performed in the ventricles.

Although the previously-described embodiments are generally directed to ablation catheters utilizing radiofrequency energy as the ablation energy sources, the mini-electrode sets described herein can be readily incorporated into catheters utilizing other ablation technologies. For example, the mini-electrode sets can be incorporated into cryoablation catheters, laser ablation catheters, ultrasound ablation catheters, and the like.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An ablation catheter comprising:
a shaft having a proximal end portion, and a distal end portion having a distal end and defining a longitudinal axis of the ablation catheter, the shaft configured to include a first deflection region at which the shaft is configured to bend in a first pre-determined direction;
an ablation electrode at the distal end of the shaft; and
a mapping region including a plurality of mini-electrode sets disposed about the shaft proximal to the ablation electrode, each of the mini-electrode sets including a plurality of mini-electrodes,
wherein the first deflection region is located proximal to or within the mapping region.

2. The ablation catheter of claim 1, wherein each of the mini-electrode sets includes a plurality of mini-electrodes arranged along a line generally parallel to the longitudinal axis, and wherein the mini-electrode sets are disposed circumferentially about the shaft.

3. The ablation catheter of claim 2, wherein the plurality of mini-electrode sets consists of three mini-electrode sets equally circumferentially spaced about the shaft.

4. The ablation catheter of claim 2, wherein the plurality of mini-electrode sets consists four mini-electrode sets equally circumferentially spaced about the shaft.

5. The ablation catheter of claim 2, wherein each of the plurality of mini-electrode sets comprises 2-8 mini-electrodes each located at a respective longitudinal position along the shaft, and wherein two of the mini-electrode sets have the same number of m ini-electrodes.

6. The ablation catheter claim 2, wherein the mini-electrodes of each mini-electrode set are longitudinally spaced at a center-to-center spacing of from 0.5 millimeters to 2 millimeters.

7. The ablation catheter of claim 2, wherein the mini-electrode sets are configured in the form of bands longitudinally spaced along the mapping region, wherein the mini-electrodes of each mini-electrode set are circumferentially spaced about the band.

8. The ablation catheter of claim 2, wherein each mini-electrode of each mini-electrode set is disposed on a flexible circuit.

9. The ablation catheter of claim 2, wherein the mini-electrodes have an active surface area of between 0.2 mm² to 1 mm².

10. The ablation catheter of claim 2, wherein the shaft further includes a second deflection region located distally of the first deflection region, the second deflection region configured to bend in a second direction different than the first direction.

11. A medical method to be performed in a patient's heart, the method comprising:
advancing an ablation electrode disposed at a distal end portion of a shaft of an ablation catheter to a position within a cardiac chamber of the patient's heart proximate to target tissue, wherein the distal end portion includes a plurality of mini-electrode sets disposed circumferentially about the shaft proximal to the ablation electrode, each of the mini-electrode sets including a plurality of mini-electrodes arranged therein;
applying ablation energy using the ablation electrode to the target tissue so as to form a conduction block within the target tissue;
after terminating the application of the ablation energy, causing at least some of the mini-electrodes to be urged into contact with the ablated tissue;
acquiring signals from the mini-electrodes in contact with the ablated tissue; and
based on the signals, analyzing the extent of the conduction block.

12. The method of claim 11, wherein the cardiac chamber is a left atrium and the target tissue is tissue proximate an ostium of a pulmonary vein of the patient's heart, and wherein the method further comprises acquiring a three-dimensional electroanatomical map of the left atrium, acquiring positional information for the distal end portion of the ablation catheter, and displaying the position of the distal end portion of the ablation catheter on the electroanatomical map during one or both of advancing the ablation electrode and applying the ablation energy.

13. The method of claim 12, further comprising updating the electroanatomical map based at least in part on the signals acquired from the mini-electrodes after terminating the ablation energy.

14. The method of claim 13, wherein causing at least some of the mini-electrodes to be urged into contact with the ablated tissue includes forming a bend in the distal end portion of the shaft.

15. The method of claim 14, wherein causing at least some of the mini-electrodes to be urged into contact with the ablated tissue further includes moving the ablation catheter so as to sweep at least some of the mini-electrodes about substantially the entire circumference of the tissue proximate the ostium.

16. The method of claim 15, further comprising re-applying ablation energy to tissue proximate the ostium if the analysis of the signals indicates a gap in the conduction block.

17. A medical system comprising:
a mapping system configured to generate a three-dimensional anatomical map of a cardiac chamber of interest;
an ablation energy source configured to provide ablation energy for a cardiac ablation procedure; and
an ablation catheter operatively coupled to the mapping system and the ablation energy source, the ablation catheter including:
a shaft having a proximal end portion, and a distal end portion having a distal end and defining a longitudinal axis of the ablation catheter, the shaft configured to include a first deflection region at which the shaft is configured to bend in a first pre-determined direction;
an ablation electrode at the distal end of the shaft operatively coupled to the ablation energy source; and
a mapping region including a plurality of mini-electrode sets disposed about the shaft proximal to the ablation electrode, wherein each of the mini-electrode sets includes a plurality of mini-electrodes operatively coupled to the mapping system,
wherein the first deflection region is located proximal to or within the mapping region.

18. The medical system of claim 17, wherein each of the mini-electrode sets includes a plurality of mini-electrodes arranged along a line generally parallel to the longitudinal axis, and wherein the mini-electrode sets are disposed circumferentially about the shaft.

19. The medical system of claim 17, wherein the mini-electrode sets are configured in the form of bands longitudinally spaced along the mapping region, wherein the mini-electrodes of each mini-electrode set are circumferentially spaced about the band.

20. The medical system of claim 17, wherein the mini-electrodes have an active surface area of between 0.2 mm² to 1 mm².
